(19) United States
(12) Patent Application Publication
(10) Pub. No.: US 2005/0208085 A1
(43) Pub. Date: Sep. 22, 2005

(54) Gelling agent

(75) Inventors: Naoya Yamato, Kawasaki-shi (JP); Seiji Takagi, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC, Tokyo (JP)

(21) Appl. No.: 11/002,117

(22) Filed: Dec. 3, 2004

Related U.S. Application Data
(63) Continuation of application No. PCT/JP03/06789, filed on May 29, 2003.

(30) Foreign Application Priority Data
Jun. 3, 2002 (JP) 161757/2002

(51) Int. Cl. A61K 7/00

(52) U.S. Cl. 424/401

(57) ABSTRACT

Gelling agents which contain at least one N-acyl-L-acidic amino acid dialkylamide represented by the following general formula (1): 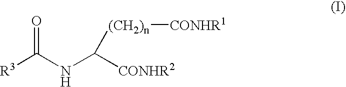
wherein R¹ and R² each independently represent a hydrocarbon group having 1 to 26 carbon atoms; R³ represents a hydrocarbon group having 7 to 10 carbon atoms; and n represents 1 or 2, such as N-2-ethylhexanoyl-L-glutamic acid dibutylamide; and at least one monohydric lower alcohol such as 3-methoxy-3-methylbutanol are useful for forming gel compositions with an oily base.

### CROSS REFERENCES TO RELATED APPLICATIONS

This application is a continuation of International Patent Application No. PCT/JP03/06789, filed on May 29, 2003, and claims priority to Japanese Patent Application No. 161757/2002, filed on Jun. 3, 2002, both of which are incorporated herein by reference in their entireties.

### BACKGROUND OF THE INVENTION

1. Field of the Invention

The present invention relates to gelling agents. More particularly, the present invention relates to gelling agents which can be dissolved in a composition at a relatively low temperature upon preparation of an oil-based gel composition, and which allow for preparation of a gel composition that is stable at high temperatures.

2. Discussion of the Background

Conventional gel aromatic substances reported hitherto include hydrated gel aromatic substances and hydrophobic gel aromatic substances having relatively low water contents, both of which have been provided in the market as commercial products. The hydrated gel aromatic substances have been known since a long time ago and include hydrous gels such as agar and carrageen, as well as polyvinyl-based and polyacrylic acid salt-based polymer hydrous gels, which have been also used recently. These are used as an aromatic substance by gradually exuding a perfume on the surface of the gel concomitant with transpiration of water. However, these hydrated gel aromatic substances are liable to water separation, and in particular, natural polymeric gels separate a large quantity of water upon thawing after freezing, thereby suffering from a marked reduction of the commercial value. Furthermore, polymer hydrous gels have low gel strength, resulting in the problem of inferiority in shape retention properties. In addition, these hydrated gel aromatic substances are poor in heat resistance, and, therefore, have been unsuitable for applications in which they are exposed to a condition of high temperature, for example, as an aromatic substance or the like for use in an automobile.

On the other hand, because the hydrophobic gel aromatic substances can readily gelatinize ethanol, ethylene glycol monoethyl ether, petroleum solvents, and natural hydrocarbon solvents, and the like with a higher fatty acid salt, particularly, sodium stearate, they have been utilized for commercial aromatic substance products (see, JP-B-S56-6783 and JP-B-S57-50502). However, with these hydrophobic gel aromatic substances, a solvent such as water, a glycol or an alcohol is an essential ingredient for dissolving sodium stearate as a gel forming agent. Thus, these hydrophobic gel aromatic substances are disadvantageous in terms of reduction of their value as a commercial product due to the instability against heat, and the produced gel being rich in residual content following preparation and also being inferior in appearance such as transparency. Examples of other hydrophobic gel aromatic substances include solid gel aromatic substances prepared with hydrogenated castor oil and a hydrocarbon compound such as d-limonene, and solid gel aromatic substances prepared with 12-hydroxystearic acid and a hydrocarbon compound such as d-limonene. However, these hydrophobic gel aromatic substances are disadvantageous in terms of even more inferior heat resistance in comparison with the afore-mentioned sodium stearate gel aromatic substance.

On the other hand, it has been disclosed that amine salts of an N-acyl amino acid, or esters or amide derivatives thereof, have a gelling performance on an oily base (see, JP-B-S53-13434). In addition, JP-B-H03-80025 discloses that a gel composition that is excellent in transparency can be obtained with the amide derivative and a volatile terpene hydrocarbon. However, according to these methods, a large quantity of a gelling agent is required for solid gelatinization of a perfume or the like, and a high temperature of 100° C. or higher is required for dissolving the gelling agent. Thus, they are not satisfactory as a method of the production of a gel composition which includes an aromatic substance.

Thus, there remains gelling agents which are free of the above-mentioned drawbacks. More particularly, there remains a need for gelling agents which can be dissolved in a composition at a relatively low temperature upon preparation of an oil-based gel composition, and which afford gel compositions that are stable at high temperatures.

### SUMMARY OF THE INVENTION

Accordingly, it is one object of the present invention to provide novel noel gelling agents.

It is another object of the present invention to provide novel gelling agents which overcome the disadvantages described above.

It is another object of the present invention to provide novel gelling agents which can be dissolved at a low temperature of 100° C. or lower to prepare an oil-based gel composition.

It is another object of the present invention to provide novel gelling agents which afford gel compositions that are stable even at high temperatures.

It is another object of the present invention to provided novel gelling agents which afford gel compositions which do not exhibit syneresis at high temperatures.

It is another object of the present invention to provide novel gelling agents which afford gel compositions that exhibit excellent in appearance.

It is another object of the present invention to provide novel gelling agents which afford gel compositions that exhibit excellent transparency.

It is another object of the present invention to provide novel gel compositions prepared from such a gelling agent.

It is another object of the present invention to provide novel gel compositions which can be utilized as an aromatic substance.

It is another object of the present invention to provide novel gel compositions which do not exhibit syneresis at high temperatures.

It is another object of the present invention to provide novel gel compositions which exhibit excellent appearance.

It is another object of the present invention to provide novel gel compositions which exhibit excellent transparency.

It is another object of the present invention to provide novel methods for making such gel compositions.

These and other objects, which will become apparent during the following detailed description, have been achieved by the inventors' discovery that a gelling agent comprising a particular N-acyl-L-acidic amino acid dialkylamide and a lower alcohol has an excellent property as a gelling agent for an oily base, can be dissolved at a low temperature of 100° C. or lower to prepare an oil-based gel composition, and allows for the preparation of a gel composition which is stable even at a condition of high temperature. Further, it has been found that the resulting gel composition is excellent in transparency and can be utilized as an aromatic substance. The invention was accomplished on the basis of the foregoing findings.

Accordingly, in a first embodiment, the present invention provides a gelling agent which comprises:

(1) at least one N-acyl-L-acidic amino acid dialkylamide represented by formula (1): 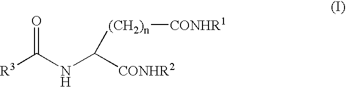
wherein R¹ and R² each independently represent a hydrocarbon group having 1 to 26 carbon atoms; R³ represents a hydrocarbon group having 7 to 10 carbon atoms; and n represents 1 or 2; and

(2) at least one monohydric lower alcohol. Such gelling agents can be used for gelatinization of an oily base.

According to one preferred embodiment of the present invention, R¹ and R² are each independently a straight chain or branched alkyl group having 1 to 26 carbon atoms; R³ is a straight chain or branched alkyl group having 7 to 10 carbon atoms; and n is 2.

According to a more preferred embodiment, R¹ and R² are each independently a straight chain or branched alkyl group having 3 to 5 carbon atoms; R³ represents a straight chain or branched alkyl group having 7 to 9 carbon atoms; and n is 2.

According to a still more preferred embodiment of the present invention, the ratio of total weight of the N-acyl-L-acidic amino acid dialkylamide represented by formula (I) to the total weight of the lower alcohol is 1:100 to 100:1.

According to a particularly preferred embodiment of the present invention, the N-acyl-L-acidic amino acid dialkylamide represented by formula (I) is N-2-ethylhexanoyl-L-glutamic acid dibutylamide. Moreover, in another preferred embodiment, the lower alcohol is 3-methoxy-3-methylbutanol or propylene glycol methyl ether.

In another aspect, the present invention provides gel compositions which comprise: (a) the afore-mentioned gelling agent and (b) at least one oily base. According to a preferred embodiment of this gel composition, the oily base is a volatile terpene hydrocarbon. In another preferred embodiment, the gel composition is suitable for use as an aromatic substance. In yet another preferred embodiment, the gel composition is a substantially transparent composition. In another preferred embodiment, the gelling agent does not exhibit substantial syneresis at high temperatures.

The present invention also provides methods of the gelatinization of an oily base, which comprises mixing a gelling agent comprising at least one compound represented by formula (I) and at least one lower alcohol with at least one oily base. The present invention also provides a method of making a gel composition comprising an oily base, which comprises mixing the afore-mentioned gelling agent with at least one oily base.

### DETAILED DESCRIPTION OF THE PREFERRED EMBODIMENTS

Thus, in a first embodiment, the present invention provides novel gelling agents which comprise:

(1) at least one N-acyl-L-acidic amino acid dialkylamide represented by formula (1): 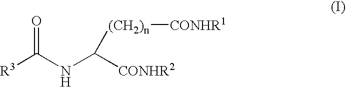
wherein R¹ and R² each independently represent a hydrocarbon group having 1 to 26 carbon atoms; R³ represents a hydrocarbon group having 7 to 10 carbon atoms; and n represents 1 or 2; and

(2) at least one monohydric lower alcohol.

In the general formula (I), R¹ and R² each independently represent a hydrocarbon group having 1 to 26 carbon atoms. The hydrocarbon group represented by R¹ and R² may be any of straight chain, branched, cyclic, or a combination thereof. Although a hydrocarbon group including an unsaturated bond can be also used as the hydrocarbon group, an alkyl group is more preferably used. A straight chain or branched alkyl group having preferably 1 to 10 carbon atoms, more preferably 2 to 6 carbon atoms may be used, and still more preferably, a straight chain or branched alkyl group having 3 to 5 atoms may be used. Most preferably, a n-butyl group may be used.

R³ represents a hydrocarbon group having 7 to 10 carbon atoms. The hydrocarbon group represented by R³ may be any of straight chain, branched, cyclic, or a combination thereof. Although a hydrocarbon group including an unsaturated bond can be also used as the hydrocarbon group, an alkyl group is more preferably used. The alkyl group is preferably a straight chain or branched alkyl group. More preferably, R³ represents a straight chain or branched alkyl group having 7 to 9 carbon atoms. Examples of the group represented by R³—CO— include, e.g., a n-octanoyl group, a n-nonanoyl group, a n-decanoyl group, a n-undecanoyl group, a 2-ethylhexanoyl group, and the like. Among them, an octanoyl group, a decanoyl group, and a 2-ethylhexanoyl group are preferred, and a 2-ethylhexanoyl group is most preferred in light of the high gelatinization capability. When the group represented by R³—CO— is a 2-ethylhexanoyl group, a 2-(R,S)-ethylhexanoyl group is preferably used in light of the ready availability of 2-ethylhexanoyl chloride which is the raw material.

In the general formula (I), the acidic amino acid residue within the molecule is an L-aspartic acid residue when n is 1, while it is an L-glutamic acid residue when n is 2. It is preferred that n is 2 in the general formula (I). The N-acyl-L-acidic amino acid dialkylamide represented by the general formula (I) may have one or more asymmetric carbons depending on the identity of R¹, R², and/or R³, and, for the gelling agent of the invention, any stereoisomer such as an optical isomer or a diastereomer based on such an asymmetric carbon, a mixture of arbitrary stereoisomers, or a racemic mixture may be also used. In addition, when R¹, R², and/or R³ have an olefinic double bond, the configuration of the same may be either Z or E. For the gelling agent of the present invention, a geometric isomer in its pure form, or any mixture of arbitrary geometric isomers may be also used. Furthermore, for the gelling agent of the present invention, an arbitrary hydrate or solvate of the N-acyl-L-acidic amino acid dialkylamide represented by the above general formula (I) may be also used.

The N-acyl-L-acidic amino acid dialkylamide represented by the general formula (I) can be prepared by, for example, producing an N-acylated glutamic acid or an N-acylated aspartic acid by subjecting a long chain fatty acid halide and L-glutamic acid or L-aspartic acid to the Schotten baumann reaction in the presence of a basic catalyst, followed by further heating the product with an amine derivative such as an alkylamine in the presence of a catalyst or in the absence of a catalyst. Alternatively, it can be produced by N-acylation of glutamic acid amide or aspartic acid amide (which is obtained by reacting glutamic acid or aspartic acid with an amine derivative such as alkylamine in the presence of an acid catalyst or in the absence of a catalyst) with an acylating agent such as a fatty acid halide.

Specific examples of the production of the N-acyl-L-acidic amino acid dialkylamide represented by the general formula (I) are explained in detail in Examples herein, and a person skilled in the art can produce any of the N-acyl-L-acidic amino acid dialkylamide represented by the general formula (I) by selecting the appropriate starting material, agents, reaction conditions, and the like with reference to the examples of production, and with making modification or alteration ad libitum to those methods as needed.

In the gelling agent of the present invention, a single compound may be used as the N-acyl-L-acidic amino acid dialkylamide represented by the general formula (I), or two or more compounds selected from the compounds represented by the general formula (I) may be used in combination. Although the amount of the N-acyl-L-acidic amino acid dialkylamide represented by the general formula (I) for use in the gelling agent of the present invention is not particularly limited, as long as it is an amount to permit gelatinization of an oily base to be gelatinized, it is usually about 0.1 to 15 parts by weight, and preferably 0.5 to 10 parts by weight, per 100 parts by weight of the oily base to be gelatinized. When the amount is less than 0.1 parts by weight, sufficient gel strength may not be achieved, while when it is greater than 15 parts by weight, the compound can not be dissolved in the oily base, which may result in an inferior appearance of the obtained gelatinized oily base.

The kind of the lower alcohol compound which may be used as the ingredient of the gelling agent of the invention is not particularly limited. Although a single compound may be used as the lower alcohol compound, two or more lower alcohol compounds may be used in combination. The term “lower” as used to herein means that the total number of carbon atoms included in the compound molecule is 10 or less, in general. Further, “alcohol compound” means a monohydric alcohol compound, without including polyhydric alcohols such as dihydric or trihydric alcohols. The lower alcohol compound may contain another arbitrary functional group. Although the kind, number, and position of the substitution of the functional group are not particularly limited, for example, one, two, or more alkoxy groups, aryl groups, oxo groups or the like may be included. Alcohol compounds in which the total number of carbon atoms within the molecule is 11 or more exhibit low volatility, and thus, when the gel composition is used as an aromatic substance, higher alcohols may remain as a residual content during or following use.

Examples of the lower alcohol compound include, e.g., methanol, ethanol, isopropyl alcohol, n-butanol, 2-butanol, t-butanol, 3-methoxybutanol, 3-methoxy-3-methylbutanol, pentanol, hexanol, cyclohexanol, cis-3-hexenol, linalool, β-phenylethyl alcohol, ethylene glycol methyl ether, ethylene glycol ethyl ether, ethylene glycol monobutyl ether, diethylene glycol monomethyl ether, diethylene glycol monoethyl ether, diethylene glycol monobutyl ether, propylene glycol methyl ether, dipropylene glycol methyl ether, propylene glycol methyl ether acetate, and the like. Among them, 3-methoxybutanol, 3-methoxy-3-methylbutanol, hexanol, cyclohexanol, ethylene glycol methyl ether, ethylene glycol ethyl ether, ethylene glycol monobutyl ether, diethylene glycol monomethyl ether, diethylene glycol monoethyl ether, diethylene glycol monobutyl ether, propylene glycol methyl ether, dipropylene glycol methyl ether, and propylene glycol methyl ether acetate are preferred. Examples of more preferred lower alcohol compounds include 3-methoxy-3-methylbutanol, ethylene glycol methyl ether, ethylene glycol ethyl ether, ethylene glycol monobutyl ether, diethylene glycol monomethyl ether, diethylene glycol monoethyl ether, diethylene glycol monobutyl ether, propylene glycol methyl ether, and dipropylene glycol methyl ether. Among them, 3-methoxy-3-methylbutanol and propylene glycol methyl ether are particularly preferred in light of possible lowering of the temperature of dissolution while keeping the suitable gelatinization capability. Although the amount of the lower alcohol compound for use is not particularly limited, for example, the compound is typically used in an amount of about 0.1 to 50 parts by weight, more preferably 1 to 20 parts by weight, per 100 parts by weight of the oily base to be gelatinized. When the amount is less than 0.1 parts by weight, the temperature for dissolution of the gelling agent may be elevated, while when it is greater than 50 parts by weight, sufficient gel strength may not be achieved.

The gelling agent of the invention comprises one, two, or more compounds selected from N-acyl-L-acidic amino acid dialkylamide represented by the general formula (I), and one, two, or more lower alcohol compounds. When this gelling agent is used, a gel composition having high gel strength and transparency, which is stable and does not exhibit substantial syneresis even at a high temperature can be produced. Although ratio of the total weight (“total weight” referred to herein means, when one kind of a compound is used, the weight of the compound, and when two or more kinds of compounds are included, the sum total weight of them) of the N-acyl-L-acidic amino acid dialkylamide represented by the general formula (I) and total weight of the lower alcohol compound may be selected ad libitum depending on the desired performances, it is preferably 1:100 to 100:1, in general, more preferably 1:50 to 50:1, even more preferably 1:10 to 10:1.

The oil for use in production of the gel composition of the present invention is not particularly limited as long as the afore-mentioned gelling agent can be sufficiently dissolved in it upon heating, and a gel can be formed when the mixture is cooled to room temperature. However, specific examples of the oil include silicone oils such as cyclomethicone and dimethicone; higher alcohols such as cetyl alcohol, isostearyl alcohol, lauryl alcohol, hexadecyl alcohol, and octyl dodecanol; fatty acids such as isostearic acid, undecylenic acid, and oleic acid; esters such as myristyl myristate, hexyl laurate, decyl oleate, isopropyl myristate, hexyldecyl dimethyloctanoate, glyceryl monostearate, diethyl phthalate, ethylene glycol monostearate, and octyl oxystearate; hydrocarbons such as d-limonene, α-pinene, liquid paraffin, Vaseline, and squalane; waxes such as lanolin, reduced lanolin, and camauba wax; fats and oils such as mink oil, cacao seed oil, coconut oil, palm kernel oil, camellia japonica oil, sesame seed oil, castor oil, and olive oil; and the like. Among them, hydrocarbon oils are preferred, and terpene hydrocarbons are more preferred in light of the appearance such as transparency of the resulting oil-based gel composition. Further, volatile terpene hydrocarbons are particularly preferred. Examples of the volatile terpene hydrocarbond include, e.g., phellandrene, terpinolene, myrcene, α-pinene, d-limonene or mixtures thereof, or natural oils comprising any of these compounds as a principal ingredient, for example, turpentine oil comprising α-pinene as a principal ingredient, orange oil comprising d-limonene as a principal ingredient, and the like.

The oily base is preferably used in a proportion of about 10 to 99% by weight per total weight of the gel composition. When the proportion of the included oily base is less than 10% by weight, or when it is greater than 99% by weight, sufficient gel strength may not be achieved. For the oily base, two or more kinds of the oily base may be used in combination.

Although the method of the production of the gel composition of the invention is not particularly limited, an intended gel composition can be obtained by, for example, heating a N-acyl-L-acidic amino acid dialkylamide, a lower alcohol compound, and an oily base to about 50 to 100° C., while stirring until the mixture forms a homogenous solution, and thereafter cooling to room temperature. In order to dissolve the gelling agent at a lower temperature, the N-acyl-L-acidic amino acid dialkylamide and the lower alcohol may be previously heated to allow dissolution, and the oil may be added when a homogenous solution is formed, followed by cooling to room temperature. Alternatively, after heating the N-acyl-L-acidic amino acid dialkylamide and the lower alcohol to allow dissolution, the solution of thus resulting mixture may be added to the oil to permit gelatinization.

For the production of the gel composition of the present invention, other gelling agent may be used in addition to the afore-mentioned gelling agent. For example, a polyamide resin which has been used as a gelling agent of an oil, a gelling agent such as 12-hydroxystearic acid, sodium stearate, aluminum octanoate, dibenzylidene-D-sorbitol, or N-lauroyl-L-glutamic acid dibutylamide may also be used. In addition, 1,2-polybutadiene or the like may be used as an auxiliary gelling agent.

To the gel composition of the present invention may be blended any ingredient such as perfumes, surfactants, various additives, various powder, and the like as needed. The particular kinds of these ingredients are not particularly limited, and such further ingredients can be selected ad libitum depending on the use or desired performance of the gel composition. Two or more kinds of these ingredients may be used in combination.

Examples of the perfume include, e.g., natural perfumes of plant origin such as rose oil, jasmine oil, neroli oil, and lavender oil; natural perfumes of animal origin such as musk oil, civet oil, and castoreum oil; synthetic hydrocarbons such as limonene and β-caryophyllene; synthetic alcohols such as cis-3-hexenol and linalool; synthetic aldehydes such as 2,6-nonadienal and citral; synthetic ketones such as β-ionone and cyclopentadecanone; synthetic esters such as linalyl acetate; synthetic lactones such as γ-undecalactone; synthetic phenols such as eugenol; synthetic oxides such as rose oxide; synthetic nitrogenous compounds such as indole; synthetic acetals such as phenylacetaldehyde dimethyl acetal; synthetic Schiff bases such as aurantiol.

Examples of the surfactant include, e.g., anionic surfactants such as N-long chain acyl amino acid salts such as N-long chain acyl acidic amino acid salts and N-long chain acyl neutral amino acid salts, N-long chain fatty acid acyl-N-methyltaurine salts, alkyl sulfates, and the alkylene oxide adducts thereof, fatty acid amide ether sulfates, metal salts and weak base salts of a fatty acid, sulfosuccinic acid surfactants, alkyl phosphates and alkylene oxide adducts thereof, alkyl ether carboxylic acids, and the like; nonionic surfactants such as ether surfactants such as glycerin ether and alkylene oxide adducts thereof, ester surfactants such as glycerin esters and alkylene oxide adducts thereof, ether ester surfactants such as sorbitan ester and alkylene oxide adducts thereof, ester surfactants such as polyoxyalkylene fatty acid esters, glycerin esters, fatty acid polyglycerin esters, sorbitan esters and sucrose fatty acid esters, alkylglucosides, hardened castor oil pyroglutamic acid diester and ethylene oxide adducts thereof, and nitrogenous nonionic surfactants such as fatty acid alkanol amides; cationic surfactants such as aliphatic amine salts such as alkyl ammonium chloride and dialkyl ammonium chloride, quaternary ammonium salts thereof, aromatic quaternary ammonium salts such as benzalkonium salts, and fatty acid acylarginine esters; and amphoteric surfactants such as betaine surfactants such as carboxy betaine, aminocarboxylic acid surfactants, and imidazoline surfactant; and the like.

Examples of the various additives include, e.g., amino acids such as glycine, alanine, serine, threonine, arginine, glutamic acid, aspartic acid, leucine, and valine; polyhydric alcohols such as glycerin, ethylene glycol, 1,3-butylene glycol, propylene glycol, and isoprene glycol; water-soluble polymers such as polyamino acids involving polyglutamic acid and polyaspartic acid, and salts thereof, polyethylene glycol, gums arabic, alginic acid salts, xanthan gum, hyaluronic acid, hyaluronic acid salts, chitin, chitosan, water soluble chitin, carboxyvinyl polymer, carboxymethyl cellulose, hydroxyethyl cellulose, hydroxypropyltrimethyl ammonium chloride, polychlorinated dimethylmethylene piperidium, polyvinyl pyrrolidone derivative quaternary ammonium, cationized proteins, collagen decomposition products and derivatives thereof, acylated proteins, and polyglycerin; sugar alcohols such as mannitol and alkyleneoxide adducts thereof; as well as animal or plant extracts; nucleic acids; vitamins; enzymes; anti-inflammatory agents; bactericides; antiseptic agents; anti-oxidizing agents; ultraviolet ray absorbing agents; chelators; antiperspirants; pigments; dyes; oxidation dyes; organic and inorganic powders; pH adjusting agents; pearl agents; wetting agents; and the like.

Examples of the various powders include, e.g., resin powders such as nylon beads and silicone beads, nylon powder; metal fatty acid soaps; yellow iron oxide; red iron oxide; black iron oxide; chromium oxide; cobalt oxide; carbon black; ultramarine blue pigments; iron blue pigments; zinc oxide; titanium oxide; zirconium oxide; silicon oxide; aluminum oxide; cerium oxide; micaceous titanium; boron nitride; barium sulfate; calcium carbonate; magnesium carbonate; aluminum silicate; magnesium silicate; silicon carbide; pigments; lakes; sericite; mica; talc; kaolin; plate-shaped barium sulfate; butterfly-shaped barium sulfate; fine particle titanium oxide; fine particle zinc oxide; fine particle iron oxide; acylamino acids; such as acyllysine, acylglutamic acid, acylarginine, and acylglycine. Furthermore, they may be subjected to a surface treatment such as a silicone treatment, a fluorine compound treatment, a silane coupling agent treatment, a silane treatment, an organic titanate treatment, an acylated lysine treatment, a fatty acid treatment, a metal soap treatment, an oil solution treatment, and an amino acid treatment.

The use of the gel composition of the present invention is not particularly limited. For example, it can be used as an aromatic substance (to impart or give off a pleasing odor), a gel cosmetic, a pack cosmetic, a granulate cosmetic, a candle or the like, and can be prepared as a composition having an appropriate form depending on the end of the same.

Other features of the invention will become apparent in the course of the following descriptions of exemplary embodiments which are given for illustration of the invention and are not intended to be limiting thereof.

### EXAMPLES

In the following examples, lauroylglutamic acid dibutylamide which was used in Comparative Example is an amino acid gelling agent “GP-1” manufactured by Ajinomoto Co., Inc.

### Example 1

### Production of N-2-(R,S)-ethylhexanoyl-L-glutamic acid dialkylamide

Sodium glutamate monohydrate in an amount of 110 g was dissolved in 140 g of water and 78 g of a 27 wt. % aqueous sodium hydroxide solution, and then cooled to 10° C. After adding 110 g of acetone, 87 g of 2-ethylhexanoyl chloride and 78 g of a 27 wt. % aqueous sodium hydroxide solution were added thereto dropwise. The acylation reaction liquid was diluted with 100 g of water and neutralized with 63 g of 95 wt. % sulfuric acid to allow separation of an oil. The water layer was removed, and the oil layer was subjected to vacuum concentration to obtain an oily substance. This oily substance was dissolved in 742 g of methanol, and 6.2 g of 95 wt. % sulfturic acid was added thereto followed by reflux for 9 hrs. After allowing the reaction liquid to cool to 35° C. and neutralizing with 8.8 g of n-butylamine, methanol was removed by distillation to obtain an oily substance. To this oily substance were added 643 g of toluene and 271 g of n-butylamine, and the mixture was heated at 90° C. for 10 hrs while stirring. Thereto were added 506 g of warm water and 130 g of 95 wt. % sulfuric acid to allow separation of an oil, and the water layer was removed. To the oil layer was added 1200 g of warm water, and the solvent was removed at atmospheric pressure to obtain a slurry solution of a white solid. This solid was collected by filtration and dried in vacuo at 50° C. to obtain 2-ethylhexanoyl glutamic acid dibutylamide.
(a) ¹³C-NMR peak (solvent: CDCl₃): 12.04, 12.07, 13.74, 13.96, 13.99, 20.08, 20.11, 22.70, 22.74, 26.01, 29.83, 31.56, 31.60, 32.37, 33.05, 39.29, 39.53, 49.37, 52.53, 52.56, 171.29, 173.03, 176.66 (ppm).
(b) ¹H-NMR peak (CDCl₃) δ: 3.248 (m, 4H), 4.373 (m, 1H), 6.199 (brs, 1H), 7.079 (brs, 1H), 7.169 (brs, 1H).
(c) wave number of infrared absorption spectrum: 3291.7, 2961.0, 2932.5, 1638.2, 1551.2, 1452.6 (cm⁻¹).
(d) MS spectrum: 382.3 (M−H)⁻

### Example 2

### Production of N-2-(R,S)-ethylhexanoyl-L-glutamic acid dibutylamide

Sodium glutamate monohydrate in an amount of 57.6 g was dissolved in 92.6 g of water, 72.9 g of isopropyl alcohol (IPA) and 41 g of a 27 wt. % aqueous sodium hydroxide solution, and then cooled to 10° C. While maintaining a pH of 11 (±0.2) and a humidity of 10 (±5)° C., 50.1 g of 2-ethylhexanoyl chloride and 49.6 g of a 27 wt. % aqueous sodium hydroxide solution were added thereto dropwise over 1.5 hrs, and thereafter, the temperature thereof was elevated to 30° C., followed by stirring for 1 hour. Thus resulting acylation reaction liquid was neutralized with 41.2 g of 75 wt. % sulfuric acid while keeping the temperature at 40° C. or lower, and the pH was adjusted to 1.9 to allow separation of an oil. The water layer was removed, and the oil layer was subjected to vacuum concentration (50° C., under reduced pressure) to obtain an oily substance. To this oily substance were added 151.9 g of water, 91.3 g of n-butanol, and 496.1 g of toluene to allow separation of an oil at 40° C. To the resulting oil layer were added 21.4 g of boron oxide and 61.9 g of butylamine, followed by reflux using an oil bath (bath temperature: 135° C.) to effect azeotropic dehydration of the thus produced water over 13 hrs. Thereto was added 444 g of diluted sulphuric acid (about 6 wt. %) to effect separation of an oil at 85° C. To the thus resulting oil layer was added 419 g of water, and separation of the oil was carried out again. Azeotropy of thus resulting oil layer was permitted under a reduced pressure while gradually adding 1000 g of water dropwise. Upon removal of n-butanol and toluene, an aqueous slurry of a white solid was obtained. The white solid was collected by filtration and dried under reduced pressure to obtain 105 g of N-2-(R,S)-ethylhexanoyl-L-glutamic acid dibutylamide (yield: 89%).

### Example 3

### Production of Gel Composition

According to the compositions shown in Table 1, 0.1 g of N-acylglutamic acid dibutylamide obtained in Example 1 was charged in an oil, with or without a lower alcohol compound. The mixture was heated in an oil bath to allow dissolution, and stood to cool to room temperature to obtain a gel composition. Further, the resulting gel composition was stored at either 0° C., room temperature, or 50° C. for one month, and the appearance thereof was visually observed, respectively. Table 1 presents the temperature required for dissolving the gelling agent, and the appearance of the gel composition. In regard to the appearance of the gel composition, a transparent solid gel was designated as 0; a transparent gel but with partial syneresis was designated as A; and a clouded gel or a completely liquefied product was designated as x.

TABLE 1

| (% by weight) | Composition 1 | Composition 2 | Composition 3 | Composition 4 | Composition 5 | Composition 6 |
|---|---|---|---|---|---|---|
| N-2-(R,S)-ethylhexanoyl-L-glutamic | 1 | 1 | 1 | 1 | | |
| acid dibutylamide | | | | | | |
| N-Lauroylglutamic acid dibutylamide | | | | | 1 | 1 |
| d-Limonene | 89.1 | 99 | 89.1 | 89.1 | 99 | 89.1 |
| 3-Methoxy-3-methylbutanol | 9.9 | | | | | 9.9 |
| Octyldodecanol | | | 9.9 | | | |
| Glycerin | | | | 9.9 | | |
| Temperature of dissolving | 95° C. or lower | 125° C. or | 95° C. or lower | 125° C. or | 110 to 115° C. | 80° C. or lower |
| gelling agent (° C.) | | higher | | higher | | |
| Appearance of sample stored at 0° C. | ◯ | ◯ | Δ | | Δ | X |
| Appearance of sample stored at room | ◯ | ◯ | Δ | X (layer | Δ | X |
| temperature | | | | separation) | | |
| Appearance of sample | ◯ | ◯ | X | | Δ | X |
| stored at 50° C. | | | | | | |

From the results shown in Table 1, it is seen that the gelling agent of the present invention can be dissolved at a relatively low temperature in an oily base when an oil-based gel composition is prepared, and the resulting gel composition is stable even at a high temperature, without causing syneresis. In addition, the resulting gel composition is also excellent in transparency and exhibits a favorable appearance (see, composition 1). On the other hand, when a lower alcohol compound was not used, the temperature of dissolving the gelling agent was greater than 100° C. (see, composition 2), and when a higher alcohol was used instead of a lower alcohol, a deterioration of the appearance after the storage at a high temperature was particularly remarkable (see, composition 3). Further, when a trihydric glycerin was used instead of a lower alcohol compound, separation of the gel composition into layers was observed (see, composition 4). Moreover, when an N-Lauroylglutamic acid dibutylamide, which is not represented by the general formula (I) was used, a deterioration of the appearance of the gel composition was also observed (see, compositions 5 and 6).

Industrial Applicability

The gelling agent of the present invention can be dissolved at a low temperature of 100° C. or lower for the preparation of an oil-based gel composition, and a stable gel composition which does not undergo syneresis even in at a high temperature can be prepared. Also, the resulting gel composition is excellent in appearance, such as transparency, and can be suitably used for applications such as an aromatic substance.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A gelling agent, which comprises:
(a) at least one N-acyl-L-acidic amino acid dialkylamide represented by formula (1): 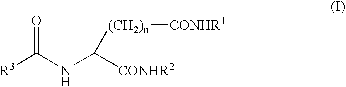
wherein R¹ and R² each independently represent a hydrocarbon group having 1 to 26 carbon atoms; R³ represents a hydrocarbon group having 7 to 10 carbon atoms; and n represents 1 or 2; and
(b) at least one monohydric lower alcohol.

2. The gelling agent of claim 1, wherein said lower alcohol comprises at least one member selected from the group consisting of 3-methoxy-3-methylbutanol, propylene glycol methyl ether, and mixtures thereof.

3. The gelling agent of claim 1, wherein R¹ and R² are each independently a straight chain or branched alkyl group having 3 to 5 carbon atoms; R³ is a straight chain or branched alkyl group having 7 to 9 carbon atoms; and n is 2.

4. The gelling agent of claim 3, wherein said lower alcohol comprises at least one member selected from the group consisting of 3-methoxy-3-methylbutanol, propylene glycol methyl ether, and mixtures thereof.

5. The gelling agent of claim 1, wherein said at least one N-acyl-L-acidic amino acid dialkylamide represented by formula (I) comprises N-2-ethylhexanoyl-L-glutamic acid dibutylamide.

6. The gelling agent of claim 5, wherein said lower alcohol comprises at least one member selected from the group consisting of 3-methoxy-3-methylbutanol, propylene glycol methyl ether, and mixtures thereof.

7. A gel composition, which comprises:
(1) at least one gelling agent according to claim 1; and
(2) at least one oily base.

8. The gel composition of claim 7, wherein said oily base comprises at least one volatile terpene hydrocarbon.

9. The gel composition of claim 7, wherein said lower alcohol comprises at least one member selected from the group consisting of 3-methoxy-3-methylbutanol, propylene glycol methyl ether, and mixtures thereof.

10. The gel composition of claim 9, wherein said oily base comprises at least one volatile terpene hydrocarbon.

11. The gel composition of claim 7, wherein R¹ and R² are each independently a straight chain or branched alkyl group having 3 to 5 carbon atoms; R³ is a straight chain or branched alkyl group having 7 to 9 carbon atoms; and n is 2.

12. The gel composition of claim 11, wherein said oily base comprises at least one volatile terpene hydrocarbon.

13. The gel composition of claim 11, wherein said lower alcohol comprises at least one member selected from the group consisting of 3-methoxy-3-methylbutanol, propylene glycol methyl ether, and mixtures thereof.

14. The gel composition of claim 13, wherein said oily base comprises at least one volatile terpene hydrocarbon.

15. The gel composition of claim 7, wherein said at least one N-acyl-L-acidic amino acid dialkylamide represented by formula (I) comprises N-2-ethylhexanoyl-L-glutamic acid dibutylamide.

16. The gel composition of claim 15, wherein said oily base comprises at least one volatile terpene hydrocarbon.

17. The gel composition of claim 15, wherein said lower alcohol comprises at least one member selected from the group consisting of 3-methoxy-3-methylbutanol, propylene glycol methyl ether, and mixtures thereof.

18. The gel composition of claim 17, wherein said oily base comprises at least one volatile terpene hydrocarbon.

19. The gel composition of claim 7, which further comprises at least one perfume.

20. A method for making a gel composition according to claim 7, said method comprising:
(a) heating a mixture of at least one N-acyl-L-acidic amino acid dialkylamide represented by formula (I), at least one lower alcohol compound, and an oily base to about 50 to 100° C., to obtain a heated mixture; and
(b) cooling said heated mixture to obtain said gel.
